(19) United States
(12) Patent Application Publication
(10) Pub. No.: US 2026/0256420 A1
(43) Pub. Date: Sep. 3, 2026

(54) DEVICE AND METHOD FOR TESTING RESPIRATORY STATE, AND DEVICE AND METHOD FOR CONTROLLING SLEEP DISORDER

(71) Applicant: Seoul National University R&DB Foundation, Seoul (KR)

(72) Inventor: Hyun-Woo SHIN, Seongnam-si (KR)

(21) Appl. No.: 19/653,800

(22) Filed: Apr. 21, 2026

Related U.S. Application Data
(63) Continuation of application No. 17/930,569, filed on Sep. 8, 2022.
Application No. 17/930,569 is a continuation of application No. PCT/KR2021/005672, filed on May 6, 2021.

(30) Foreign Application Priority Data
May 6, 2020 (KR) 10-2020-0054051
Aug. 14, 2020 (KR) 10-2020-0102803
Sep. 29, 2020 (KR) 10-2020-0127093

(51) Int. Cl. A61B 5/00

(52) U.S. Cl. CPC A61B 5/4818; A61B 5/7267; A61B 5/7289; A61B 5/7425; A61B 5/743

(57) ABSTRACT

A respiratory status examination apparatus and method, and a sleep disorder control device and method are proposed. The apparatus may include at least one image capturing unit that is movably arranged to adjust a distance with respect to a subject and configured to obtain a thermal image by photographing the subject. The apparatus may also include a motion sensor unit configured to detect a motion of the subject to generate motion information, and a temperature information extracting unit configured to specify at least one examination region from the thermal image obtained by the image capturing unit and extract temperature information from the examination region. The apparatus may further include a respiratory status examining unit configured to determine a respiratory status of the subject based on the temperature information extracted by the temperature information extracting unit and the motion information generated by the motion sensor unit.

### CROSS REFERENCE TO RELATED APPLICATIONS

This application is a continuation of U.S. patent application Ser. No. 17/930,569, filed Sep. 8, 2022, which is a continuation application of International Application No. PCT/KR 2021/005672, filed on May 6, 2021, which claims the benefit of Korean Patent Application Nos. 10-2020-0054051 filed on May 6, 2020, 10-2020-0102803 filed on Aug. 14, 2020, and 10-2020-0127093 filed on Sep. 29, 2020 in the Korean Intellectual Property Office, the entire disclosure of each of which is incorporated herein by reference.

### BACKGROUND

### Technical Field

Embodiments of the present disclosure relate to a respiratory status examination apparatus and method, and a sleep disorder control device and method.

### Description of Related Technology

In general, when the muscles surrounding the airway are relaxed during sleep, the uvula, tonsils, and tongue collapse backwards. This may result in a slightly narrower airway than when awake, but this is not a problem for most people. However, for some people, snoring or obstructive sleep apnea (OSA) occurs because the airways are severely narrowed during sleep by this phenomenon, preventing air from passing through the airways.

### SUMMARY

One aspect is a respiratory status monitoring apparatus and method, whereby a respiratory status of a patient may be easily and precisely examined while alleviating discomfort of the patient.

Embodiments of the present disclosure provide a sleep disorder control device and method for maximizing the mandibular advancement effect.

Embodiments of the present disclosure are intended to provide a polysomnography device which allows efficient learning by using processed images, instead of time-series data of a source signal of examination units as learning data, and an examination method thereof.

A respiratory status monitoring apparatus according to an embodiment of the present disclosure may include: at least one image capturing unit that is movably arranged to adjust a distance with respect to a subject and configured to obtain a thermal image by photographing the subject; a motion sensor unit configured to detect a motion of the subject to generate motion information; a temperature information extracting unit configured to specify at least one examination region from the thermal image obtained by the image capturing unit and extract temperature information from the examination region; and a respiratory status examining unit configured to determine a respiratory status of the subject based on the temperature information extracted by the temperature information extracting unit and the motion information generated by the motion sensor unit.

According to a respiratory status monitoring apparatus and method according to embodiments of the present disclosure, a decrease in the accuracy of examination due to obstruction factors may be prevented by capturing a thermal image by using a near-infrared or infrared camera, and the discomfort of a subject may be reduced through a non-contact type examination method.

According to a sleep disorder control device and an operating method thereof according to embodiments of the present disclosure, a sleep disorder may be detected using biometric information, and when treating the detected sleep disorder by advancing the mandible, arousal due to the movement of the mandible may be minimized by also considering a sleep satisfaction level of a user to thereby improve sleep quality.

According to the sleep disorder control device and the operating method thereof according to the embodiments of the present disclosure, not only sleep satisfaction level data obtained immediately after a sleep but also sleep satisfaction level data of before going to sleep (which data evaluating daytime activity or cognitive ability, etc.) after spending a day may be used as learning data, and thus the learning efficiency may be improved.

According to a polysomnography device and an examination method thereof according to embodiments of the present disclosure, instead of raw data obtained from a plurality of examination units, a graph image generated using the raw data is used as learning data, and thus, accurate reading results may be derived while increasing the efficiency of artificial intelligence-or deep learning-based learning.

The polysomnography device and the examination method thereof according to the embodiments of the present disclosure may realize automated examination through a trained sleep state reading model, thereby shortening the examination time as well as reducing the examination deviation according to readers.

### BRIEF DESCRIPTION OF THE DRAWINGS

FIG. 1 illustrates a respiratory status monitoring apparatus according to an embodiment of the present disclosure.

FIG. 2 illustrates a respiratory status monitoring apparatus according to another embodiment of the present disclosure.

FIG. 3 illustrates a respiratory status monitoring apparatus according to another embodiment of the present disclosure.

FIG. 4 illustrates a processor and a motion sensor unit of the respiratory status monitoring apparatus according to the present disclosure.

FIG. 5 illustrates a method of specifying an examination region and extracting temperature information, performed by the respiratory status monitoring apparatus according to the present disclosure.

FIG. 6 illustrates a method of adjusting a position of an image capturing unit of the respiratory status monitoring apparatus according to the present disclosure.

FIG. 7 is a flowchart illustrating a respiratory status monitoring method according to an embodiment of the present disclosure in order.

FIG. 8 is a diagram schematically illustrating a mandibular advancement system according to an embodiment of the present disclosure.

FIG. 9 is a block diagram schematically illustrating a server according to an embodiment of the present disclosure.

FIGS. 10 and 11 are diagrams for explaining a process of obtaining and learning sleep satisfaction level data.

FIG. 12 is a flowchart sequentially illustrating a sleep disorder control method according to an embodiment of the present disclosure.

FIG. 13 is a flowchart for explaining a control method of the mandibular advancement system.

FIG. 14 is a block diagram schematically illustrating a polysomnography device 100″ according to an embodiment of the present disclosure.

FIG. 15 is a conceptual diagram for explaining a process of obtaining polysomnography data from a plurality of examination units.

FIG. 16 shows a graph image which is learning data of a polysomnography device according to an embodiment of the present disclosure.

FIG. 17 shows a labeled graph image.

FIG. 18 is a diagram sequentially illustrating an examination method of a polysomnography device according to an embodiment of the present disclosure.

### DETAILED DESCRIPTION

Snoring or obstructive sleep apnea (OSA) can lower the quality of a person's sleep or cause other complex problems, and thus an examination and treatment are required, and thus apparatuses and methods for examination and treating symptoms are developed. However, the devices and methods developed and used so far cause discomfort or pain of the patient during the examination or treatment, preventing good quality sleep, and furthermore, the precision or accuracy of the examination thereof is low. Therefore, development of a technique enabling precise diagnosis and observation of snoring or OSA of a patient and allowing alleviation of the discomfort of a user in a treatment process is required.

A respiratory status monitoring apparatus according to an embodiment of the present disclosure may include: at least one image capturing unit that is movably arranged to adjust a distance with respect to a subject and configured to obtain a thermal image by photographing the subject; a motion sensor unit configured to detect a motion of the subject to generate motion information; a temperature information extracting unit configured to specify at least one examination region from the thermal image obtained by the image capturing unit and extract temperature information from the examination region; and a respiratory status examining unit configured to determine a respiratory status of the subject based on the temperature information extracted by the temperature information extracting unit and the motion information generated by the motion sensor unit.

In an embodiment of the present disclosure, the image capturing unit may be provided in plurality, and the plurality of the image capturing units may be arranged apart from each other around the subject.

In an embodiment of the present disclosure, the image capturing unit may include a near-infrared camera.

In an embodiment of the present disclosure, the temperature information extracting unit may specify a plurality of examination regions from the thermal image, and the plurality of the examination regions may include: a first examination region specified based on positions of the nose and mouth of the subject; a second examination region specified based on positions of the chest and abdomen of the subject; and a third examination region specified based on positions of the arms and legs of the subject.

In an embodiment of the present disclosure, the respiratory status examining unit may determine the respiratory status of the subject based on the temperature information detected from the first examination region to the third examination region.

In an embodiment of the present disclosure, the respiratory status monitoring apparatus may further include a learning unit that learns, by machine learning, respiratory status determination criteria based on the temperature information and the motion information, wherein the respiratory status examining unit determines the respiratory status of the subject based on the respiratory status determination criteria.

In an embodiment of the present disclosure, the respiratory status monitoring apparatus may further include a position adjuster adjusting a position of the image capturing unit according to a change in a posture of the subject.

In an embodiment of the present disclosure, the respiratory status monitoring apparatus may further include a learning unit that learns, by machine learning, posture determination criteria for determining the posture of the subject, based on the motion information, wherein the position adjuster determines the posture of the subject based on the posture determination criteria, and adjusts the position of the image capturing unit according to the determined posture of the subject.

In an embodiment of the present disclosure, a respiratory status monitoring method may include: capturing a thermal image of a subject by using a near-infrared camera; specifying, by a temperature information extracting unit, an examination region from the thermal image, based on positions of the nose and mouth of the subject; extracting, by the temperature information extracting unit, temperature information from the examination region; generating, by a motion sensor unit, motion information by detecting a motion of the subject; and detecting, by a respiratory status examining unit, a respiratory status of the subject based on the temperature information and the motion information.

In an embodiment of the present disclosure, the near-infrared camera may be provided in plurality, and the plurality of the near-infrared cameras may be arranged apart from each other around the subject.

In an embodiment of the present disclosure, the respiratory status monitoring method may further include specifying, by the temperature information extracting unit, an additional examination region; and detecting, by the temperature information extracting unit, temperature information from the additional examination region, wherein the additional examination region is specified based on at least one of positions of the chest and abdomen of the subject and positions of arms and legs of the subject.

In an embodiment of the present disclosure, the respiratory status monitoring method may further include learning, by a learning unit by machine learning, respiratory status determination criteria based on the temperature information and the motion information.

In an embodiment of the present disclosure, the detecting of the respiratory status of the subject may include determining the respiratory status of the subject based on the respiratory status determination criteria.

In an embodiment of the present disclosure, the respiratory status monitoring method may further include adjusting, by a position adjuster, a position of the near-infrared camera according to a change in a posture of the subject.

In an embodiment of the present disclosure, the respiratory status monitoring method may further include learning, by a learning unit by machine learning, posture determination criteria based on the motion information, wherein the adjusting of the position of the near-infrared camera includes determining, by the position adjuster, the posture of the subject based on the posture determination criteria.

An embodiment of the present disclosure provides a sleep disorder control method including: obtaining sleep satisfaction level data and bio-signal data of a user wearing a sleep disorder treatment device, and usage record data of the sleep disorder treatment device; training a machine learning model based on the sleep satisfaction level data, the bio-signal data, and the usage record data; and controlling an operation of the sleep disorder treatment device while the user is wearing the sleep disorder treatment device, by using the sleep satisfaction level data, the bio-signal data, the usage record data, and the machine learning model.

In an embodiment of the present disclosure, the obtaining the sleep satisfaction level data and the bio-signal data of the user and the usage record data of the sleep disorder treatment device may include obtaining the bio-signal data of the user and the usage record data of the sleep disorder treatment device during a sleep of the user wearing the sleep disorder treatment device and obtaining the sleep satisfaction level data after the user wearing the sleep disorder treatment device completes the sleep.

In an embodiment of the present disclosure, the obtaining of the sleep satisfaction level data may include obtaining first sleep satisfaction level data at a first time point when the user completes sleep and obtaining second sleep satisfaction level data at a second time point different from the first time point.

In an embodiment of the present disclosure, the obtaining of the second sleep satisfaction level data may obtain the second sleep satisfaction level data after a preset period of time from the first time point and before a next sleep of the user.

In an embodiment of the present disclosure, the obtaining of the sleep satisfaction level data may include generating a first notification signal to the user before the first time point and generating a second notification signal to the user before the second time point.

In an embodiment of the present disclosure, the controlling of the operation of the sleep disorder treatment device may include controlling a degree of advancement or the number of advances of the sleep disorder treatment device while the user is wearing the sleep disorder treatment device.

An embodiment of the present disclosure provides a sleep disorder control device including: a data obtaining unit configured to obtain sleep satisfaction level data and bio-signal data of a user wearing a sleep disorder treatment device, and usage record data of the sleep disorder treatment device; a learning unit configured to train, by machine learning, a machine learning model, based on the sleep satisfaction level data, the bio-signal data, and the usage record data; and an operation controller configured to control an operation of the sleep disorder treatment device while the user is wearing the sleep disorder treatment device, by using the sleep satisfaction level data, the bio-signal data, the usage record data, and the machine learning model.

In an embodiment of the present disclosure, the data obtaining unit may include: a bio-signal obtaining unit configured to obtain the bio-signal data by using one or more sensors during a sleep of the user wearing the sleep disorder treatment device; a usage record obtaining unit configured to obtain the usage record data of the sleep disorder treatment device during the sleep of the user wearing the sleep disorder treatment device; and a sleep satisfaction level obtaining unit configured to obtain the sleep satisfaction level data after the user wearing the sleep disorder treatment device completes the sleep.

In an embodiment of the present disclosure, the sleep satisfaction level obtaining unit may obtain first sleep satisfaction level data at a first time point when the user completes the sleep and second sleep satisfaction level data at a second time point different from the first time point.

In an embodiment of the present disclosure, the second sleep satisfaction level data may be obtained at the second time point which is after a preset period of time from the first time point and before a next sleep of the user.

In an embodiment of the present disclosure, the sleep disorder control device may further include a notification signal generator that generates a first notification signal to the user before the first time point and generates a second notification signal to the user before the second time point.

In an embodiment of the present disclosure, the operation controller may control, by using the sleep satisfaction level data, the bio-signal data, the usage record data, and the machine learning model, a degree of advancement or the number of advances of the sleep disorder treatment device while the user is wearing the sleep disorder treatment device.

An embodiment of the present disclosure provides a polysomnography device including: a graph image generator configured to obtain polysomnography raw data that is measured in time series, and convert the polysomnography data into a graph with respect to time to generate a graph image, a learning unit configured to train a sleep state reading model based on the graph image; and a reader configured to read a sleep state of a user based on the graph image and the sleep state reading model.

In an embodiment of the present disclosure, the polysomnography device may further include: a split image generator configured to generate a plurality of images by splitting the graph image in units of a preset time, wherein the learning unit trains the sleep state reading model based on the plurality of images obtained by the splitting of the graph image.

In an embodiment of the present disclosure, the polysomnography data may be a plurality of pieces of biometric data of a user, which are measured using a plurality of examination units, and the graph image generator may convert each piece of the plurality of biometric data into an individual graph with respect to time, and sequentially arrange the converted, plurality of individual graphs on a time axis to generate the graph image.

In an embodiment of the present disclosure, the plurality of pieces of biometric data may include biometric data obtained using at least one of sensing units among an Electroencephalogram (EEG) sensor, an Electrooculography (EOG) sensor, an Electromyogram (EMG) sensor, an Electrokardiogramme (EKG) sensor, a Photoplethysmography (PPG) sensor, a chest belt, an abdomen belt, oxygen saturation, end-tidal CO2 (EtCO2), a respiration detection thermistor, a flow sensor, a pressure sensor (manometer), a microphone, and a positive pressure gauge of a continuous positive pressure device.

In an embodiment of the present disclosure, the graph image generator may generate the graph image by matching times of the plurality of pieces of biometric data.

In an embodiment of the present disclosure, the graph image may include labeled data.

An embodiment of the present disclosure provides an examination method of a polysomnography device, the method including: obtaining time-serially measured polysomnography data; converting the polysomnography data into a graph with respect to time to generate a graph image; training a sleep state reading model based on the graph image; and reading a sleep state of a user based on the graph image and the sleep state reading model.

In an embodiment of the present disclosure, the method may further include generating a plurality of images by splitting the graph image in units of a preset time, and the training of the sleep state reading model may include training the sleep state reading model based on the plurality of images obtained by the splitting.

In an embodiment of the present disclosure, the polysomnography data may include a plurality of pieces of biometric data of a user, which are measured using a plurality of examination units, and the graph image generator may convert each of the plurality of pieces of biometric data into an individual graph with respect to time, and sequentially arrange the converted, plurality of individual graphs on a time axis to generate the graph image.

In an embodiment of the present disclosure, the plurality of pieces of biometric data may include biometric data obtained using at least one of sensing units among an Electroencephalogram (EEG) sensor, an Electrooculography (EOG) sensor, Electromyogram (EMG) sensor, an Electrokardiogramme (EKG) sensor, a Photoplethysmography (PPG) sensor, a chest belt, an abdomen belt, oxygen saturation, end-tidal CO2 (EtCO2), a respiration detection thermistor, a flow sensor, a pressure sensor (manometer), a microphone, and a positive pressure gauge of a continuous positive pressure device.

In an embodiment of the present disclosure, the generating of the graph image may include generating the graph image by matching times of the plurality of pieces of biometric data.

In an embodiment of the present disclosure, the generating of the graph image may include generating the graph image including labeled data.

Other aspects, features and advantages other than those described above will become apparent from the following drawings, claims, and detailed description of the present disclosure.

As the present disclosure allows for various changes and numerous embodiments, particular embodiments will be illustrated in the drawings and described in detail in the written description. However, this is not intended to limit the present disclosure to particular modes of practice, and it is to be appreciated that all changes, equivalents, and substitutes that do not depart from the spirit and technical scope of the present disclosure are encompassed in the present disclosure. In the description of the present disclosure, certain detailed explanations of related art are omitted when it is deemed that they may unnecessarily obscure the essence of the present disclosure.

While such terms as “first,” “second,” etc., may be used to describe various components, such components must not be limited to the above terms. The above terms are used only to distinguish one component from another.

The terms used in the present specification are merely used to describe particular embodiments, and are not intended to limit the present disclosure. An expression used in the singular encompasses the expression of the plural, unless it has a clearly different meaning in the context. In the drawings, each constituent element is exaggerated, omitted, or schematically illustrated for convenience of explanation and clarity, and the size of each constituent element does not perfectly reflect an actual size.

In the description of each constituent element, when each constituent element is described as being formed “on” or “under” a constituent element, the constituent element may be formed “directly” or “indirectly” with any other constituent element interposed therebetween “on” or “under” the constituent element. The status of “on” or “under” of a constituent element is described based on the drawings.

Hereinafter, embodiments of the present disclosure will be described below in more detail with reference to the accompanying drawings. Those components that are the same or are in correspondence are rendered the same reference numeral regardless of the figure number, and redundant explanations are omitted.

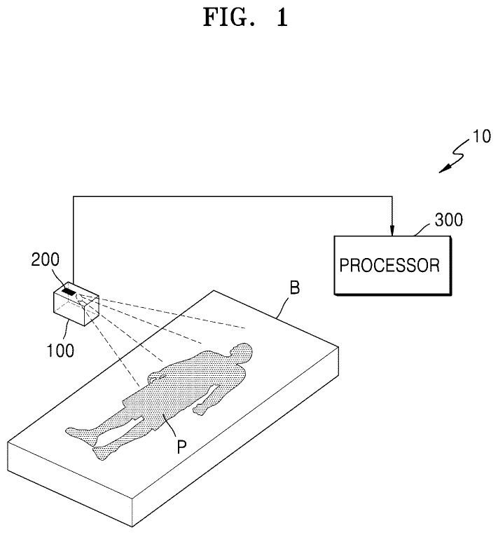

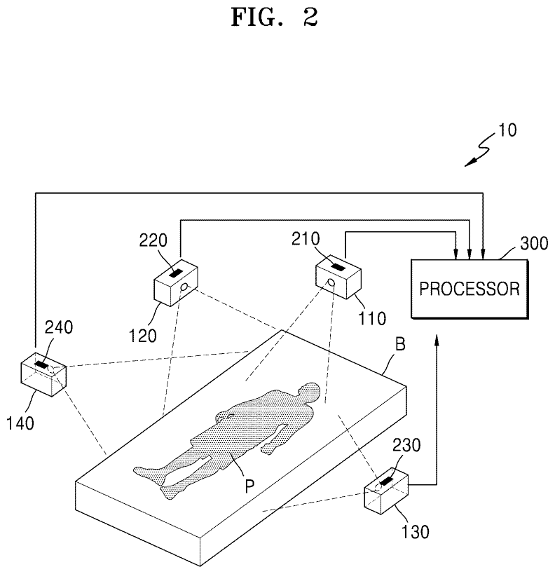

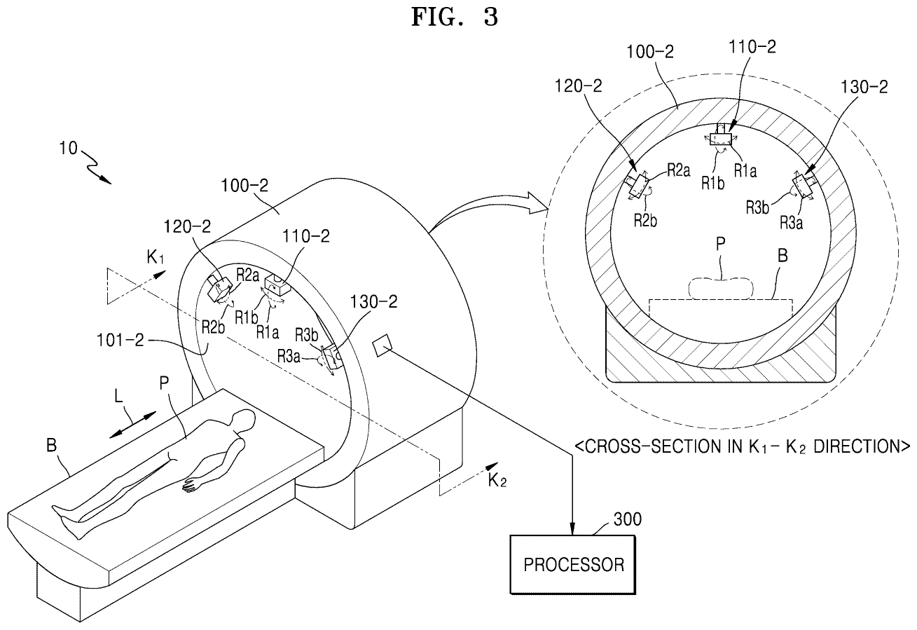

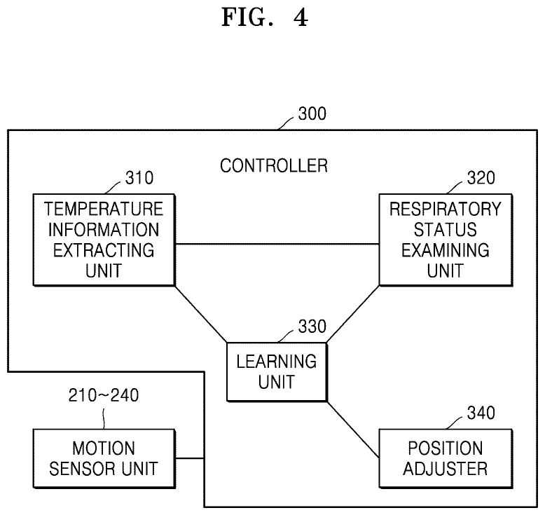

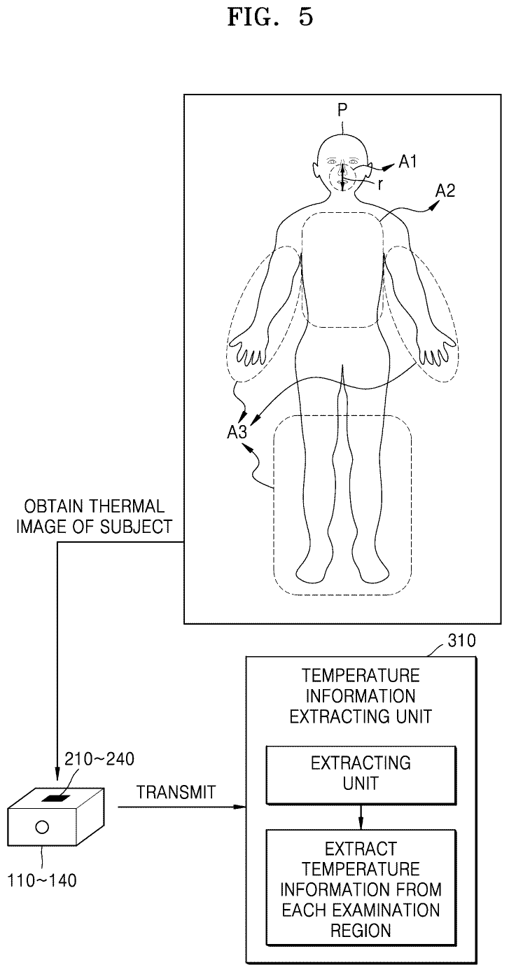

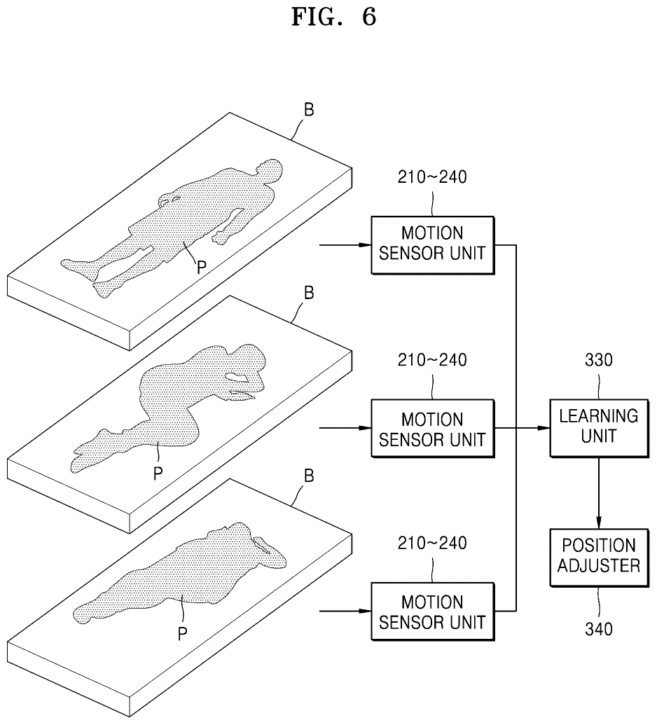

FIG. 1 illustrates a respiratory status monitoring apparatus according to an embodiment of the present disclosure. FIG. 2 illustrates a respiratory status monitoring apparatus according to another embodiment of the present disclosure, and FIG. 3 illustrates a respiratory status monitoring apparatus according to another embodiment of the present disclosure. FIG. 4 illustrates a processor and a motion sensor unit according to the present disclosure. FIG. 5 illustrates a method of specifying an examination region and extracting temperature information, performed by the respiratory status monitoring apparatus according to the present disclosure. FIG. 6 illustrates a method of adjusting a position of an image capturing unit of the respiratory status monitoring apparatus according to the present disclosure.

Referring to FIGS. 1 to 6, a respiratory status monitoring apparatus 10 may include an image capturing unit 100, a motion sensor unit 200, a temperature information extracting unit 310, and a respiratory status examining unit 320. In addition, the respiratory status monitoring apparatus 10 may further include a position adjuster 340 and a learning unit (or a learning processor) 330.

The respiratory status may include a normal respiratory status, a hypopnea state, and an apnea state, and it may be determined, based on a change in body temperature of a subject P, which state a current respiratory status of the subject P corresponds to. For example, during exhalation, the temperature around the nose and mouth of the subject P may rise as air heated by the body temperature of the subject P is discharged to the outside through the nose and mouth. Accordingly, a thermal image of the subject P, captured by a thermal imaging camera, and a temperature signal of the subject P may be changed. Here, compared with the degree of change in a thermal image and a temperature signal in a normal respiratory status, for example, the degree of change in the thermal image and the temperature signal may be lower in a hypopnea state, and in another example, there may be no change in a thermal image of the surroundings in the case of apnea. Accordingly, a respiration-specific pattern may be determined by analyzing thermal images of the vicinity of the nose and mouth.

The image capturing unit 100 may capture an image of the subject P to obtain a thermal image of the subject P. The image capturing unit 100 may include a thermal imaging camera capable of photographing a temperature distribution of the body of the subject P. As an embodiment, the thermal imaging camera may include a near-infrared camera, an infrared camera, or other cameras capable of capturing a thermal image of a human body. However, for convenience, description below will focus on an embodiment in which the image capturing unit 100 includes a near-infrared camera. As the image capturing unit 100 includes a near-infrared camera, the image capturing unit 100 may obtain a thermal image of the subject P without being disturbed by obstacles even when there is an interference factor between the image capturing unit 100 and the subject P (e.g., a blanket covering the subject P, clothes that the subject P is wearing, a curtain arranged between the subject P and the image capturing unit 100, etc.). In this case, a thermal image captured by the image capturing unit 100 may be, for example, a near-infrared multi-spectral image.

The image capturing unit 100 may be arranged apart from the subject P. In this case, the image capturing unit 100 may be spaced apart, by a certain distance, from the subject P or an examination bed B on which the subject P is located, and thus may capture an image of the subject P while not contacting the subject P.

The image capturing unit 100 may be movably arranged. In this case, the image capturing unit 100 may adjust a distance from the image capturing unit 100 to the subject P. Accordingly, by adjusting a position of the image capturing unit 100 according to the body characteristics such as the height of the subject P, a required thermal image of a body region of the subject P may be obtained.

At least one image capturing unit 100 may be included. In an embodiment, as illustrated in FIG. 1, one image capturing unit 100 may be included. In this case, the image capturing unit 100 may be arranged at an optimal position for obtaining a thermal image of the subject P. For example, the image capturing unit 100 may be located above the subject P or the examination bed B on which the subject P is located, and here, the image capturing unit 100 may be located above the tiptoe of the subject P or above the head of the subject P. As another example, the image capturing unit 100 may be located around the examination bed B with respect to the examination bed B, and in this case, the image capturing unit 100 may be arranged at a side of the subject P or at a side of the examination bed B.

In another embodiment, as illustrated in FIG. 2, a plurality of image capturing units 100 may be included. In this case, the plurality of image capturing units 100 may be arranged apart from each other. In detail, the plurality of image capturing units 100 may be arranged apart from each other in a circumferential direction of the subject P or the examination bed B, with respect to the subject P or the examination bed B. In an embodiment, the plurality of image capturing units 100 may include a first image capturing unit 110, a second image capturing unit 120, a third image capturing unit 130, and a fourth image capturing unit 140. Here, the first image capturing unit 110 to the fourth image capturing unit 140 may be respectively arranged at different positions from each other, that is, adjacent to one of an upper end (e.g., the head of the subject P, a right side, a left side, and a lower end (e.g., the tiptoe of the subject P of the examination bed B. In this case, the first image capturing unit 110 to the fourth image capturing unit 140 may respectively obtain thermal images captured in various directions and at various angles by capturing images of the subject P at different positions and angles. By capturing thermal images at various positions by using the plurality of image capturing units 100, noise of the thermal images may be removed and the reliability of thermal imaging results may be improved.

As another embodiment, as illustrated in FIG. 3, an image capturing unit 100-2 may capture a thermal image of the subject P while linearly moving in a longitudinal direction (for example, L direction in FIG. 3) of the examination bed B on which the subject P is located. Here, the image capturing unit 100-2 may have a moving hole 101-2 which is arranged therein and through which the examination bed B passes. The image capturing unit 100-2 may have a disk shape. However, the present disclosure is not limited thereto, and the image capturing unit 100-2 may have various shapes such as a square plate, a polygonal plate, and the like.

The image capturing unit 100-2 may include a camera 110-2. The camera 110-2 may be arranged on an inner surface of the image capturing unit 100-2. The camera 110-2 is rotatable about a connection shaft connected to the inner surface 102-2 of the image capturing unit 100-2, and a tilting angle of the camera 110-2 may be adjusted. In this case, a position of the camera 110-2 may be changed while the camera 110-2 is rotated based on movement of the subject P detected by a motion sensor unit.

As an embodiment, the image capturing unit 100-2 may include a plurality of cameras. The number of the plurality of cameras is not limited, but for convenience of description, description will focus on an embodiment in which the image capturing unit 100-2 includes three cameras (that is, a first camera 110-2, a second camera 120-2, and a third camera 130-2).

The first camera 110-2, the second camera 120-2, and the third camera 130-2 may be spaced apart from each other along a circumferential direction of the image capturing unit 100-2, and arranged on the inner surface 102-2 of the image capturing unit 100-2. For example, the first camera 110-2 may be arranged on the inner surface 102-2 of the image capturing unit 100-2, in parallel to an arbitrary line that is parallel to the longitudinal direction of the examination bed B and passes through a center of the examination bed B, and the second camera 120-2 and the third camera 130-2 may be arranged symmetrically with respect to the first camera 110-2. In this case, the subject P on the examination bed B moving through the moving hole 101-2 of the image capturing unit 100-2 may be photographed at different angles.

As described above, the first camera 110-2, the second camera 120-2 and the third camera 130-2 may rotate about the connection shaft connected to the image capturing unit 100-2. Here, since each of the first camera 110-2 to the third camera 130-2 is rotated independently of each other, a rotation direction and a tilt angle of each camera may be different from each other. For example, the first camera 110-2 is rotatable in a direction R1a or a direction R1b, the second camera 120-2 is rotatable in a direction R2a or a direction R2b, and the third camera 130-2 is rotatable in a direction R3a or a direction R3b. Accordingly, by measuring thermal images of the subject P from various angles, and synthesizing these thermal images to evaluate the respiratory status of the subject P, the accuracy of examination may be improved.

The motion sensor unit 200 may generate motion information by detecting a motion of the subject P. The motion information may be information including a movement path and movement position of at least one of a body part of the subject P and the entire body of the subject P. In an embodiment, when the subject P moves a body part such as a face, arm, or leg, the motion sensor unit 200 may detect a motion of the body part, and track the motion to detect a movement path and a movement position of the body part. As another embodiment, the motion sensor unit 200 may detect a motion of each body part of the subject P, and detect a movement path and a movement position of each body part by tracking the motion, or may detect a movement path and a movement position of the whole body of the subject P based on the detected movement paths and movement positions of the respective body parts. The motion sensor unit 200 may generate a motion signal showing a movement of the subject P, such as the movement path and the movement position detected as described above.

A plurality of motion sensor units 200 may be provided. In this case, the plurality of motion sensor units 200 may be arranged apart from each other. The motion sensor units 200 may be arranged apart from each other along the circumferential direction of the subject P or the examination bed B, with respect to the subject P or the examination bed B. In an embodiment, the plurality of motion sensor units 200 may include a first motion sensor unit 210, a second motion sensor unit 220, a third motion sensor unit 230, and a fourth motion sensor unit 240. Here, the first motion sensor unit 210 to the fourth motion sensor unit 240 may be respectively arranged at different positions from each other, that is, adjacent to one of the upper end (e.g., the head of the subject P, the right side, the left side, and the lower end (e.g., the tiptoe of the subject P of the examination bed B. In this case, each of the first motion sensor unit 210 to the fourth motion sensor unit 240 may generate motion information by detecting a motion of the subject P at different positions and angles, thereby precisely determining the motion of the subject P and improving the reliability of the generated motion information.

The motion sensor unit 200 may transmit the generated motion information to the respiratory status examining unit 320 or the learning unit 330.

The respiratory status monitoring apparatus 10 according to an embodiment of the present disclosure may include one or more processors 300. The respiratory status monitoring apparatus 10 may be driven in a form included in a hardware device, such as a microprocessor or a general-purpose computer system. Here, the ‘processor’ may refer to, for example, a data processing device embedded in hardware and having a physically structured circuit to perform a function expressed as code or a command included in a program. Examples of the data processing device embedded in hardware as described above may include a microprocessor, a central processing unit (CPU), a processor core, a multiprocessor, an application-specific integrated circuit (ASIC), and a field programmable gate array (FPGA), but the present disclosure is not limited thereto. The processor 300 may include the temperature information extracting unit 310 and the respiratory status examining unit 320. In addition, the processor 300 may further include the learning unit 330 and the position adjuster 340.

The temperature information extracting unit 310 may receive a thermal image captured by the image capturing unit 100 and specify an examination region A based on the received thermal image. The examination region A may be a portion or a region of the body in which a change in body temperature of the subject P may be checked in order to determine the respiratory status of the subject P.

The temperature information extracting unit 310 may specify at least one examination region A. As an embodiment, the temperature information extracting unit 310 may specify one examination region A. In this case, the one examination region A may be specified to include an optimal position for determining the respiratory status of the subject P. For example, the examination region A may be specified based on the positions of the nose and mouth of the subject P; in this case, as illustrated in FIG. 5, the temperature information extracting unit 310 may set an imaginary circle having, as a diameter, a straight line connecting from the nose to the jaw of the subject P, and specify an inner region of the imaginary circle as the examination region A.

As another embodiment, a plurality of examination regions A may be specified. For example, the plurality of examination regions A may include a first examination region A1, a second examination region A2, and a third examination region A3. The first examination region A1 may be specified to include the nose and mouth of the subject P, and in this case, the method of specifying the first examination region A1 may be the same as described above. The second examination region A2 may be specified based on the positions of the chest and abdomen of the subject P. For example, the second examination region A2 may be specified as a region extending from just below the clavicle of the subject P, through the chest and abdomen, to the top of the pelvis. The third examination region A3 may be specified based on the positions of the arms and legs of the subject P. However, the present disclosure is not limited thereto, and the number and positions of the examination regions A may be changed according to parts to be examined, which require temperature information extraction.

The temperature information extracting unit 310 may extract temperature information from the specified examination region A. The temperature information may include a body temperature or an amount of change in the body temperature in the examination region A, extracted from the thermal image. When the subject P breathes, the body temperature of the subject P in the examination region A may change. For example, when the subject P breathes in (inhalation), the temperature of the nose, mouth, and the skin surface in the vicinity thereof of the subject P may drop, and when the subject P exhales (exhalation), the temperature of the nose, mouth, and the skin surface in the vicinity thereof of the subject P may rise. As another example, when there is a motion of the subject P, the body temperature of a body part of the subject P may rise.

The temperature information may further include information about the amount of change in carbon dioxide and water vapor in the examination region A in each case of inhalation and exhalation of the subject P. Near-infrared rays have a wavelength of 0.78 μm to 3 μm, and can penetrate to a depth of several millimeters from the skin surface of the subject P, and atmospheric components that absorb infrared rays may vary depending on wavelength bands in the atmosphere. For example, at around 4.3 microns, infrared rays are absorbed by carbon dioxide, and at around 6.5 microns, infrared rays may be absorbed by water vapor, whereby near infrared rays may be selectively transmitted. Due to this selective transmittance of near-infrared rays, the relative amounts of carbon dioxide and water vapor in the examination region A may be significantly vary depending on the wavelength of near-infrared rays during inhalation and exhalation of the subject P. For example, during exhalation of the subject P, the amount of carbon dioxide and water vapor in the examination region A may increase in a certain wavelength band compared to inhalation. The amount of change in carbon dioxide and water vapor in the examination region A according to the wavelength of the near-infrared rays may be analyzed and used to detect the respiratory status of the subject P.

In an embodiment in which a plurality of examination regions A are specified, the temperature information extracting unit 310 may extract temperature information from each of the plurality of examination regions A. For example, the temperature information extracting unit 310 may extract a temperature and/or a temperature change amount of the nose, mouth, and the surrounding areas thereof in the first examination region A1, a temperature and/or a temperature change amount of the chest, abdomen, and the surrounding areas thereof in the second examination region A2, and a temperature and/or a temperature change amount of the arms and legs and the surrounding areas thereof in the third examination region A3, respectively. In addition, the temperature information extracting unit 310 may segment the body of the subject P by increasing the number of specified examination regions A, thereby making it possible to determine a temperature change in each body part and thus allowing to selectively detect temperature information of only certain body parts that needs examination.

The temperature information extracting unit 310 may extract temperature information from the examination region A and transmit the same to the respiratory status examining unit 320 or the learning unit 330.

The respiratory status examining unit 320 may determine the respiratory status of the subject P based on temperature information and motion information. In this case, the respiratory status examining unit 320 may measure a body temperature and motion of the subject P in real time, and monitor the respiration volume, respiratory status, and sleep state or the like of the subject P in real time.

In an embodiment, the learning unit 330 may learn respiratory status determination criteria of the subject P by machine learning. The learning unit 330 may learn, by machine-learning, the respiratory status determination criteria on the basis of at least one of temperature information received from the temperature information extracting unit 310 and motion information received from the motion sensor unit 200. In another embodiment, the learning unit 330 may learn, by machine-learning, posture determination criteria of the subject P. Here, the learning unit 330 may learn, by machine learning, posture determination criteria on the basis of the motion information received from the motion sensor unit 200. For example, the learning unit 330 may learn the respiratory status determination criteria or the posture determination criteria by using a machine learning or deep-learning method.

The position adjuster 400 may adjust a position of the image capturing unit 100 according to a posture of the subject P. The position adjuster 400 may determine the posture of the subject P based on the posture determination criteria learned by the learning unit 330. Here, the position adjuster 400 may determine the posture of the subject P by applying information such as the movement path and movement position of the subject P or of body parts of the subject P, measured by the motion sensor unit 200, to the posture determination criteria.

After determining the posture of the subject P, the position adjuster 400 may adjust the position or a photographing angle of the image capturing unit 100 according to the determined posture.

As an embodiment, the position adjuster 400 may adjust a tilting angle of the image capturing unit 100 or rotate the image capturing unit 100 according to the determined posture. As another embodiment, the position adjuster 400 may adjust the position of the image capturing unit 100 by moving the image capturing unit 100 up, down, left and right around the subject P or the examination bed B according to the determined posture. As another embodiment, the position adjuster 340 may adjust the tilting angle and the position of the image capturing unit 100 differently depending on whether the determined posture is a supine, lateral or prone position. In this case, the image capturing unit 100 may capture an image of the subject P at a position adjusted by the position adjuster 400, and the respiratory status examining unit 320 may determine the respiratory status of the subject P based on the captured image, and thus, even if the posture of the subject P is changed during monitoring, the examination may be continuously performed with uniform accuracy.

When the processor 300 includes the learning unit 330, the respiratory status examining unit 320 may determine the respiratory status of the subject P based on the respiratory status determination criteria. In this case, the respiratory status examining unit 320 may monitor the respiration volume, the respiratory status, and sleep state of the subject P in real time by measuring a change in body temperature and motion of the subject P in real time and applying the learned respiratory status determination criteria.

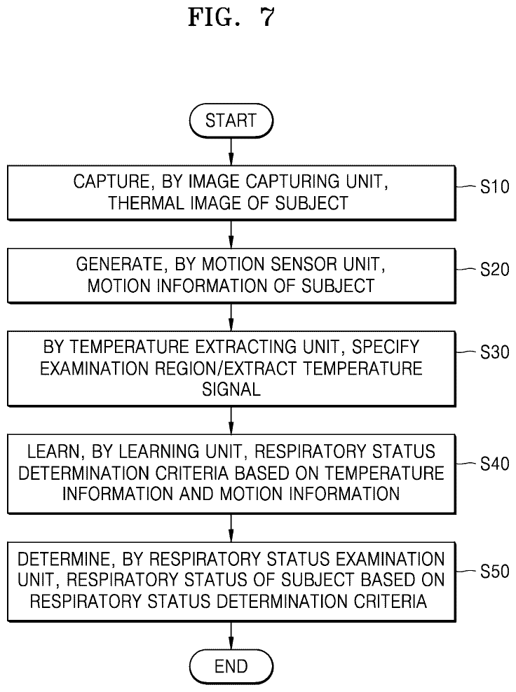

FIG. 7 is a flowchart of a respiratory status monitoring method according to an embodiment of the present disclosure in order.

Referring to FIG. 7, the respiratory status monitoring method according to an embodiment of the present disclosure is as described below, and hereinafter, description will focus on an embodiment in which the processor 300 includes the learning unit 330 and the position adjuster 340.

In operation S10, the image capturing unit 100 may capture a thermal image of the subject P. In this case, the image capturing unit 100 may obtain a thermal image of the subject P by using a near-infrared camera or an infrared camera. Here, the image capturing unit 100 may include a plurality of near-infrared cameras or infrared cameras, and the plurality of near-infrared or infrared cameras may be spaced apart from each other and arranged at different positions to obtain thermal images in various directions and from various angles.

In operation S20, the motion sensor unit 200 may generate motion information by detecting a motion of the subject P. The motion sensor unit 200 may detect a motion of the subject P or a motion of a certain body part of the subject P, and track the motion and generate motion information based on a movement path and movement position of the subject P or the certain body part of the subject P.

In operation S30, the temperature information extracting unit 310 may specify the examination region A from the thermal image captured by the image capturing unit 100, based on the positions of the nose and mouth of the subject P. Next, the temperature information extracting unit 310 may extract temperature information from the specified examination region A. As an embodiment, the temperature information extracting unit 310 may specify an additional examination region and extract temperature information from the additional examination. The additional examination region may be specified based on at least one of the positions of the chest and abdomen and the positions of arms and legs of the subject P.

In operation S40, the learning unit 330 may learn, by machine learning, the respiratory status determination criteria on the basis of the temperature information extracted from the temperature information extracting unit 310 and the motion information generated by the motion sensor unit 200. As another embodiment, the learning unit 330 may learn, by machine learning, the posture determination criteria of the subject P on the basis of the motion information generated by the motion sensor unit 200.

In operation S50, the respiratory status examining unit 320 may detect the respiratory status of the subject P based on the temperature information extracted from the temperature information extracting unit 310 and the motion information generated by the motion sensor unit 200. Here, the respiratory status examining unit 320 may determine the respiration volume, the respiratory status, and the lifespan state of the subject P based on the learned respiratory status determination criteria.

The position adjuster 400 may adjust a position of the image capturing unit 100 according to a posture of the subject P. A method, by the position adjuster 400, of adjusting the position of the image capturing unit 100 may be as follows.

The position adjuster 400 may first determine the posture of the subject P based on the posture determination criteria learned by the learning unit 330. Here, the position adjuster 400 may determine the posture of the subject P by applying, to the posture determination criteria, information such as the movement path and movement position of the subject P or of the body part of the subject P, measured by the motion sensor unit 200.

Next, the position adjuster 400 may adjust a position of the image capturing unit 100 according to the determined posture of the subject P. In an embodiment, the position adjuster 400 may adjust the tilting angle of the image capturing unit 100 according to the determined posture, or adjust the image capturing unit 100 by rotating the image capturing unit 100. In another embodiment, the position adjuster 400 may adjust the position of the image capturing unit 100 by moving the image capturing unit 100 up, down, left and right with respect to the subject P or the examination bed B, according to the determined posture.

Next, the respiratory status monitoring apparatus may capture a thermal image of the subject P at the adjusted position of the image capturing unit 100, and perform again the examination operation described above.

As described above, according to the respiratory status monitoring apparatus and method according to the embodiments of the present disclosure, a decrease in the accuracy of examination due to obstruction factors may be prevented by taking a thermal image by using a near-infrared or infrared camera, and the discomfort of the subject may be reduced through a non-contact type examination method.

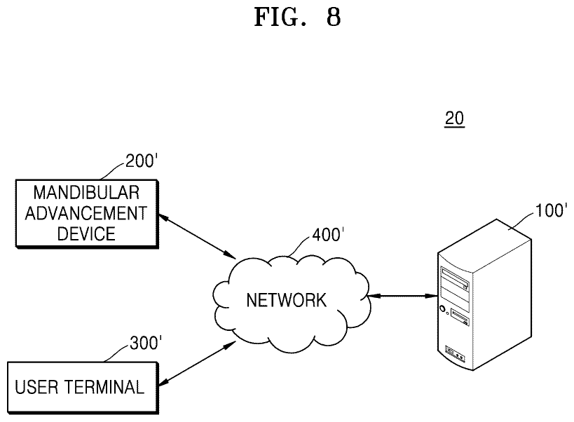

FIG. 8 is a diagram schematically illustrating a sleep disorder treatment system 20 according to an embodiment of the present disclosure.

Referring to FIG. 8, the sleep disorder treatment system 20 according to an embodiment of the present disclosure includes a sleep disorder control device 100′, a sleep disorder treatment device 200′, a user terminal 300′, and a network 400′.

The sleep disorder treatment system 20 according to an embodiment of the present disclosure may detect a bio-signal of a user while the user is wearing the sleep disorder treatment device 200′ and sleeping, and move the mandible or adjust positive pressure according to a sleep state of the user, determined using the detected bio-signal, thereby alleviating sleep disorders such as snoring or apnea in a customized manner. Here, the sleep disorder treatment system 20 may obtain not only the bio-signal but also user sleep satisfaction level data, and train a machine learning model based on bio-signal data and sleep satisfaction level data, to thereby minimize arousal of the user during sleep and thus improve sleep quality.

The sleep disorder control device 100′ may include a server implemented using a computer device that communicates with the sleep disorder treatment device 200′0 and the user terminal 300′ to provide commands, code, files, contents, services, etc., or a plurality of the computer devices. However, the present disclosure is not limited thereto, and the sleep disorder control device 100′ may be integrally formed with the sleep disorder treatment device 200′.

For example, the sleep disorder control device 100′ may provide a file for installing an application to the user terminal 300′ accessed through the network 400′. In this case, the user terminal 300′ may install an application by using the file provided from the sleep disorder control device 100′. In addition, according to the control by an operating system (OS) and at least one program (e.g., a browser or an installed application) included in the user terminal 300′, the sleep disorder control device 100′ may be accessed to receive services or contents provided by the sleep disorder control device 100′. As another example, the sleep disorder control device 100′ may establish a communication session for data transmission or reception, and route data transmission or reception between the user terminals 30′ through the established communication session.

The sleep disorder control device 100′ may include a processor, obtain user sleep satisfaction level data and bio-signal data and train a machine learning model based on deep learning, and perform a function of controlling the sleep order treatment device 200′ by using the machine learning model. However, the present disclosure is not limited thereto, and after training the machine learning model through the sleep disorder control device 100′, the machine learning model may be provided to the sleep disorder treatment device 200′ for the sleep disorder treatment device 200′ to determine a degree of mandibular advancement or the number of advances. Hereinafter, for convenience of description, an embodiment in which learning and control are performed in the server 100′ will be mainly described.

The sleep disorder treatment device 200′ refers to a treatment unit that a user can wear for treatment of a sleep disorder during sleep. The sleep disorder treatment device 200′ may be, for example, a mandibular advancement device for advancing the mandible, or a positive pressure device for controlling air pressure. Also, although not described, the sleep disorder treatment device 200′ may be applied to any treatment unit that the user may wear while sleeping. Hereinafter, for convenience of description, description will focus on a case in which the sleep disorder treatment device 200′ is a mandibular advancement device.

The sleep disorder treatment device 200′ may include an upper teeth seating portion and a lower teeth seating portion that are arranged in the oral cavity, a driving unit advancing or withdrawing the lower teeth seating portion, relative to the upper teeth seating portion, and a sensing unit for sensing a bio-signal of the user, to move the lower jaw of the user based on a sleep state while the user is wearing the sleep disorder treatment device 200′. In addition, the sleep disorder treatment device 200′ may include a communicator that transmits bio-signal data sensed through the sensor unit, to the user terminal 300′ or the sleep disorder control device 100′.

The upper teeth seating portion may be a potion on which the user's upper teeth are seated. The upper teeth seating portion may be formed in a shape into which the user's upper teeth may be inserted. The upper tooth seating portion may be customized according to the user's teeth in order to minimize the foreign body sensation or discomfort when the upper teeth are seated thereon. When the upper teeth seating portion is worn on the upper teeth, the upper teeth seating portion may wrap and be closely adhered to the upper teeth.

The lower teeth seating portion may be a potion on which the user's lower teeth are seated. The lower tooth seating portion may be customized according to the user's teeth in order to minimize the foreign body sensation or discomfort when the lower teeth are seated thereon. When the lower teeth seating portion is worn on the lower teeth, the lower teeth seating portion may wrap and be closely adhered to the lower teeth.

The driving unit may be connected to the upper teeth seating portion and the lower teeth seating portion to change a relative position of the lower teeth seating portion with respect to the upper teeth seating portion. The driving unit may include a power unit providing a driving force and a power transmission unit transmitting the driving force generated by the power unit, to the upper teeth seat portion or the lower teeth seat portion.

The sensing unit may detect biometric information of the user. The sensing unit may include various sensors that detect biometric information for determining whether the user is sleeping, a posture, or a sleep state, such as snoring, or sleep apnea. For example, the sensing unit may include at least one of a respiration sensor, an oxygen saturation sensor, and a posture sensor.

The respiration sensor may be an acoustic sensor capable of detecting a snoring sound, or an airflow sensor detecting respiration of the user, inhaled or exhausted through the nose or mouth. The oxygen saturation sensor may be a sensor for detecting oxygen saturation. Here, the respiration sensor and the oxygen saturation sensor may obtain a bio-signal for determining a sleep state such as snoring or sleep apnea of the user.

The posture sensor may be a sensor that detects a bio-signal for determining a sleeping posture of the user. The posture sensor may consist of a single component, but may also include different types of sensors arranged at different positions to obtain biometric information. For example, the posture sensor may include a three-axis sensor. The three-axis sensor may be a sensor that detects changes in a yaw axis, a pitch axis, and a roll axis. The three-axis sensor may include at least one of a gyro sensor, an acceleration sensor, and a tilt sensor. In addition, the present disclosure is not limited thereto, and a sensor for detecting changes in axes of a number different from three may also be applied.

The communicator may include a communication unit capable of communicating with the sleep disorder control device 100′ or the user terminal 300′, for example, Bluetooth, ZigBee, Medical Implant Communication Service (MISC), or Near Field Communication (NFC). The communicator may transmit bio-signal data sensed through the sensing unit to the user terminal 300′ or the sleep disorder control device 100′.

The user terminal 300′ may be a stationary terminal implemented as a computer device or a mobile terminal. The user terminal 300′ may be a terminal of an administrator who controls the sleep disorder control device 100′. Alternatively, the user terminal 300′ may be an obtaining unit for obtaining sleep satisfaction level data of the user through an interface. The user terminal 300′ may display questionnaire information for obtaining a level of sleep satisfaction provided by the sleep disorder control device 100′, and generate sleep satisfaction level data by using the questionnaire information selected by the user. The user terminal 300′ may include, for example, a smart phone, a mobile phone, a navigation system, a computer, a laptop computer, a digital broadcasting terminal, a personal digital assistant (PDA), a portable multimedia player (PMP), a tablet PC, and the like. As an example, the user terminal 300′ may communicate with another user terminal 300′, the sleep disorder treatment device 200′, or the sleep disorder control device 100′ through the network 400′ by using a wireless or wired communication method.

The communication method is not limited, and not only a communication method using a communication network that the network 400′ may include (e.g., a mobile communication network, a wired Internet, a wireless Internet, a broadcasting network), but also short-range wireless communication between devices may be included as the communication method. For example, the network 400′ may include one or more of a personal area network (PAN), a local area network (LAN), a controller area network (CAN), a metropolitan area network (MAN), a wide area network (WAN), a metropolitan area network (MAN), a wide area network (WAN), a broadband network (BBN), and the Internet. Further, the network 400′ may include one or more of network topologies including a bus network, a star network, a ring network, a mesh network, a star-bus network, a tree or a hierarchical network, and the like, but is not limited thereto.

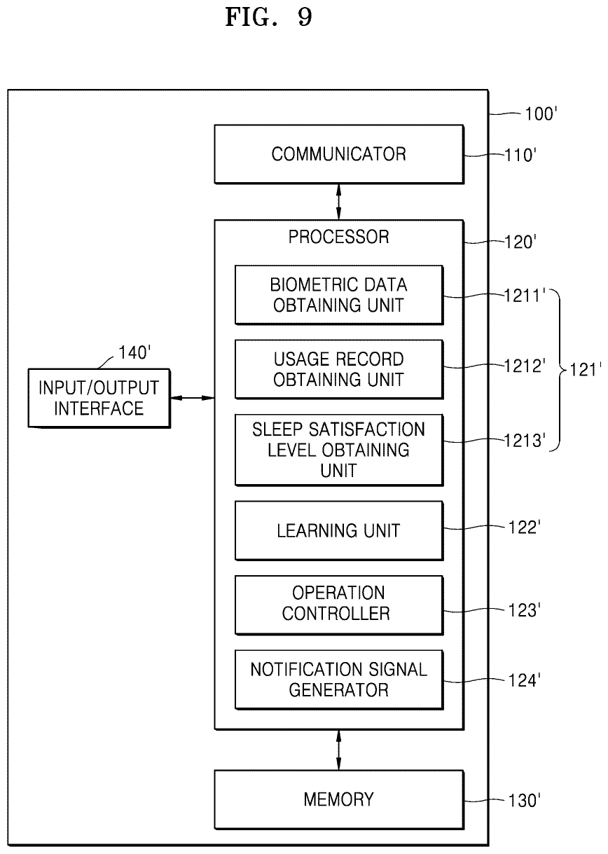

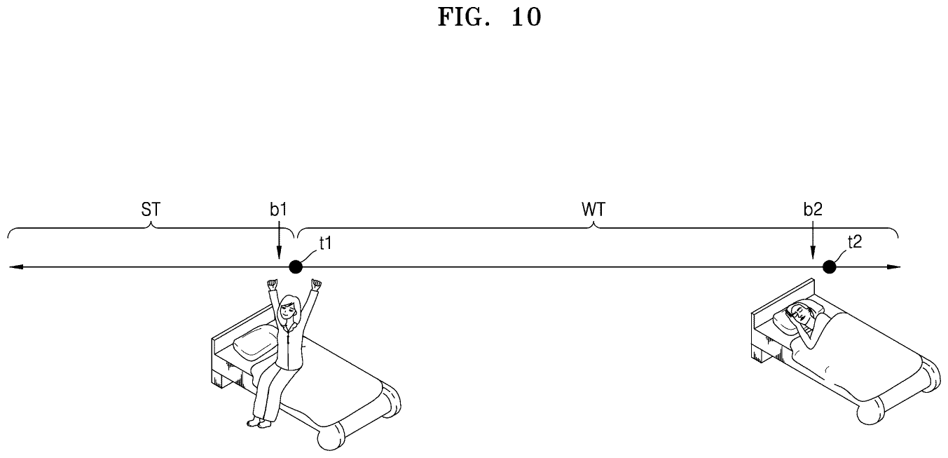

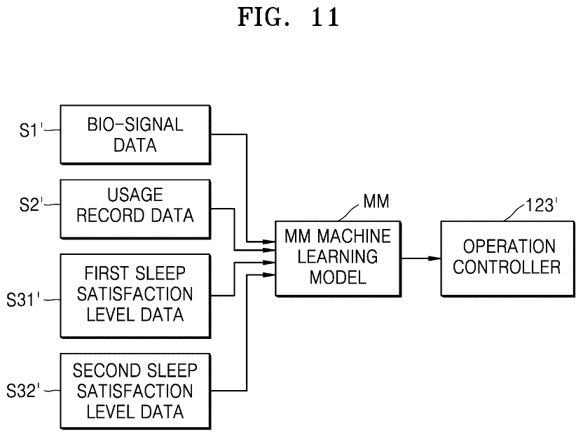

FIG. 9 is a block diagram schematically illustrating the sleep disorder control device 100′ according to an embodiment of the present disclosure, and FIGS. 10 and 11 are diagrams for explaining a process of obtaining and learning sleep satisfaction level data.

Referring to FIGS. 9 to 11, the sleep disorder control device 100′ according to an embodiment of the present disclosure may include a communicator 110′, a processor 120′, a memory 130′, and an input/output interface 140′.

The communicator 110′ may receive bio-signal data and usage record data from the sleep disorder treatment device 200′, or may receive sleep satisfaction level data from the user terminal 300′. The communicator 110′ may receive bio-signal data S1′ and usage record data S2′ during a sleep period ST of the user wearing the sleep disorder treatment device 200′. The communicator 110′ may receive sleep satisfaction level data S3′ during an awake period WT after the user completes sleep.

The processor 120′ may be configured to process a command of a computer program by performing basic arithmetic, logic, and input/output operations. The command may be provided to the processor 120′ by the memory 130′ or the communicator 110′. For example, the processor 120′ may be configured to execute the received command according to program code stored in a recording device, such as the memory 130′. Here, the ‘processor’ may refer to, for example, a data processing device embedded in hardware and having a physically structured circuit to perform a function expressed as code or a command included in a program.

Examples of the data processing device embedded in hardware as described above may include a microprocessor, a central processing unit (CPU), a processor core, a multiprocessor, an application-specific integrated circuit (ASIC), and a field programmable gate array (FPGA), but the present disclosure is not limited thereto. The processor 120′ may include a data obtaining unit 121′, a learning unit 122′, an operation controller 123′, and a notification signal generator 124′.

The data obtaining unit 121′ may obtain the sleep satisfaction level data S3′ and the bio-signal data S1′ of the user wearing the sleep disorder treatment device 200′ and the usage record data S2′ of the sleep disorder treatment device 200′. The data obtaining unit 121′ may include a biometric data obtaining unit 1211′, a usage record obtaining unit 1212′, and a sleep satisfaction level obtaining unit 1213′.

The biometric data obtaining unit 1211′ may obtain the bio-signal data S1′ by using one or more sensors during sleep of the user wearing the sleep disorder treatment device 200′. Here, the bio-signal data S1′ may be data generated by the sensing unit of the sleep disorder treatment device 200′. For example, the bio-signal data S1′ may include information about a respiration volume, snoring sound information, and posture information detected through a respiration sensor, an oxygen saturation sensor, and a posture sensor. The biometric data obtaining unit 1211′ may receive bio-signal data sensed in real time during the user's sleep period ST. However, the present disclosure is not limited thereto, and the biometric data obtaining unit 1211′ may receive the bio-signal data S1′ when a sleep apnea event occurs, or receive the bio-signal data S1′ according to a preset cycle.

The usage record obtaining unit 1212′ may obtain the usage record data S2′ of the sleep disorder treatment device 200′ during a sleep of the user wearing the sleep disorder treatment device 200′. Here, the usage record data S2′ may be a history of driving the sleep disorder treatment device 200′ by using the bio-signal data S1′, for example, a time at which the mandible was advanced overnight, a total period of time that the mandible was advanced, the number of advances, the degree of advances, and the like.

The usage record obtaining unit 1212′ may obtain the usage record data S2′ from the sleep disorder treatment device 200′, but the present disclosure is not limited thereto, and the usage record data S2′ may also be obtained through a control signal generated by the operation controller 123′ to be described later.

The sleep satisfaction level obtaining unit 1213′ may obtain the sleep satisfaction level data S3′ after the user who is wearing the sleep disorder treatment device 200′ completes sleep. Here, the sleep satisfaction level data S3′ may be obtained by providing questionnaire information including a sleep satisfaction-related questionnaire through the interface of the user terminal 300′ and by using the user's response information with respect to the questionnaire information. The sleep satisfaction level data S3′ may be data obtained by quantifying sleep satisfaction level by using the user's response information. The sleep satisfaction-related questionnaire may be a questionnaire about whether sleep was satisfactory, or the user has a morning headache, emotional changes and depression, concentration, and a dry throat. The sleep satisfaction level data S3′ may include not only response information on the sleep satisfaction level, but also personal information of the user. The sleep satisfaction level data S3′ may further include personal information such as the age, gender, height, and weight of the user.

The sleep satisfaction level obtaining unit 1213′ may obtain one or more pieces of sleep satisfaction level data S3′. In detail, the sleep satisfaction level obtaining unit 1213′ may obtain first sleep satisfaction level data S31′ at least at a first time point t1 when the user completes sleep. That is, the sleep satisfaction level obtaining unit 1213′ may obtain data about the user's sleep satisfaction level immediately after sleep. Also, the sleep satisfaction level obtaining unit 1213′ may obtain second sleep satisfaction level data S32′ at a second time point t2 different from the first time point t1. The second time point t2 may be after a preset period of time from the first time point t1 and before a next sleep of the user, and the user may enter state information about daytime sleepiness, concentration, work efficiency, etc. through the user terminal 300′.

In another embodiment, the sleep satisfaction level obtaining unit 1213′ may obtain sleep satisfaction level data not only at the time point t1 immediately after waking up and at the time point t2 just before falling asleep, but also at other preset time points. For example, the sleep satisfaction level obtaining unit 1213′ may additionally obtain sleep satisfaction level data after eating lunch.

The learning unit 122′ may train a machine learning model MM based on the obtained sleep satisfaction level data S3′, the obtained bio-signal data S1′, and the obtained usage record data S2′. The machine learning model MM may be an algorithm for learning control criteria for controlling operation of the sleep disorder treatment device based on the sleep satisfaction level data S3′, the bio-signal data S1′, and the usage record data S2′.

The sleep disorder treatment device 200′ may detect a sleep disorder such as sleep apnea, through the bio-signal data S1′, and perform a function of treating the disorder by advancing the mandible, and the operation of advancing the mandible may cause inevitable arousal and decrease sleep quality. The sleep disorder treatment system 20 according to an embodiment of the present disclosure may perform a function of improving sleep quality by not only advancing the mandible simply based on a bio-signal but also by minimizing the number of advances of the mandible in consideration of sleep satisfaction level.

To this end, the learning unit 122′ may learn the control criteria for controlling the operation of the sleep disorder treatment device 200′ by using the sleep satisfaction level data S3′, the bio-signal data S1′, and the usage record data S2′. In detail, the learning unit 122′ may learn control criteria for controlling the degree of advance or the number of advances of the sleep disorder treatment device 200′.

In addition, the learning unit 122′ may learn the control criteria regarding in which of cases where the mandible is to be selectively advanced, by using the bio-signal data S1′ of the sleep disorder treatment device 200′. When it is determined that the user has fallen into light sleep, the learning unit 122′ may train, by using the bio-signal data S1′, the machine learning model such that the mandible is not advanced.

The learning unit 122′ trains a machine learning model based on deep learning or artificial intelligence, and deep learning is defined by a machine learning algorithm that tries high-level abstractions (summarizing key contents or functions from large amounts of data or complex data) through a combination of non-linear transformation methods. The learning unit 122′ may use, among deep learning models, for example, one of a deep neural network (DNN), a convolutional neural network (CNN), a recurrent neural network (RNN), and a deep belief neural network (DBN).

The operation controller 123′ may control the operation of the sleep disorder treatment device 200′ by using the bio-signal data S1′, the usage record data S2′, the sleep satisfaction level data S3′, and the machine learning model MM while the user is wearing the sleep disorder treatment device 200′. The operation controller 123′ may control the number of advances or the degree of advances of the mandibular advancement device by applying new bio-signal data S1′, the usage record data S2′, and the sleep satisfaction level data S3′ to the trained machine learning model MM.

The notification signal generator 124′ may provide a first notification signal b1 to the user before the first time point t1 and provide a second notification signal b2 to the user before the second time point t2. When the notification signal generator 124′ provides the first notification signal b1 and the second notification signal b2 to the user terminal 300′, the user terminal 300′ may notify, through sound, vibration, a screen or light, the user that it is time to respond to sleep satisfaction level. The notification signal generator 124′ may generate the first notification signal b1 within a preset period of time from immediately after the user wakes up, and the second notification signal b2 at a preset time point before the average time that user falls asleep.

The memory 130′ is a computer-readable recording medium and may include a random access memory (RAM), a read only memory (ROM), and a permanent mass storage device such as a disk drive. In addition, the memory 130′ may store an operating system and at least one program code (e.g., code for a browser installed and driven in a user terminal or the application described above). These software components may be loaded from a computer-readable recording medium that is readable by an additional computer, separate from the memory 130′, by using a drive mechanism. The computer-readable recording medium readable by an additional computer may include a computer-readable recording medium such as a floppy drive, a disk, a tape, a DVD/CD-ROM drive, and a memory card. In another embodiment, the software components may be loaded into the memory 130′ through the communicator′ instead of a computer-readable recording medium. For example, at least one program may be loaded to the memory 130′ based on a program (e.g., the application described above) installed by files provided by, through a network, a file distribution system (e.g., the server described above) for distributing installation files of developers or applications.

The input/output interface 140′ may be used for interfacing with an input/output device. For example, an input device may include a device such as a keyboard or mouse, and an output device may include a device such as a display for displaying a communication session of an application. As another example, the input/output interface 140″ may be used for interfacing with a device in which functions for inputting and outputting are integrated into one, such as a touch screen.

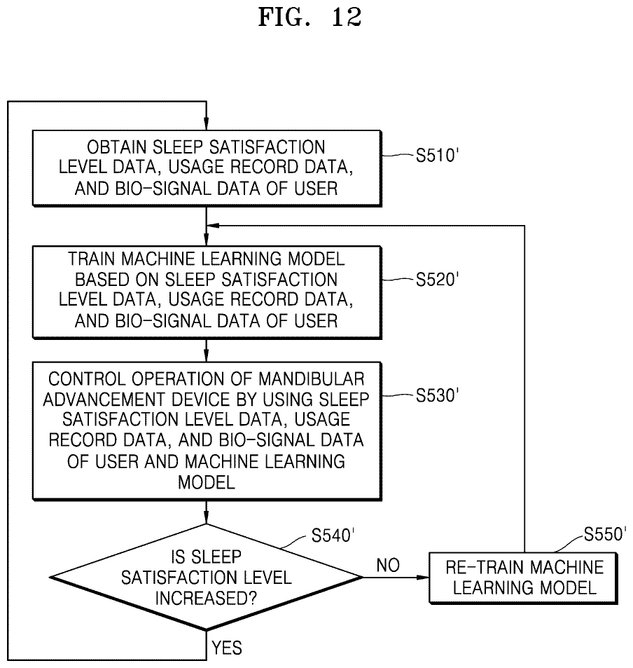

FIG. 12 is a flowchart sequentially illustrating a sleep disorder control method according to an embodiment of the present disclosure.

Referring to FIG. 12, the server 100′ may obtain, by using a data obtaining unit, sleep satisfaction level data and bio-signal data of a user who wears the sleep disorder treatment device 200′, and usage record data of the sleep disorder treatment device 200′ in operation S510′.

Next, the server 100′ may train a machine learning model based on the sleep satisfaction level data, the bio-signal data, and the usage record data, by using a learning unit in operation S520′.

Next, the server 100′ may control, by using an operation controller, the operation of the sleep disorder treatment device 200′ while the user is wearing the same, by using the sleep satisfaction level data, the bio-signal data, the usage record data, and the machine learning model. The sleep disorder control device 100′ may control whether or not the sleep disorder treatment device 200′ is advanced, an advanced distance, an advancing speed, an advancing force, or the number of advances to thereby minimize unnecessary arousal of the user so as to improve sleep quality.

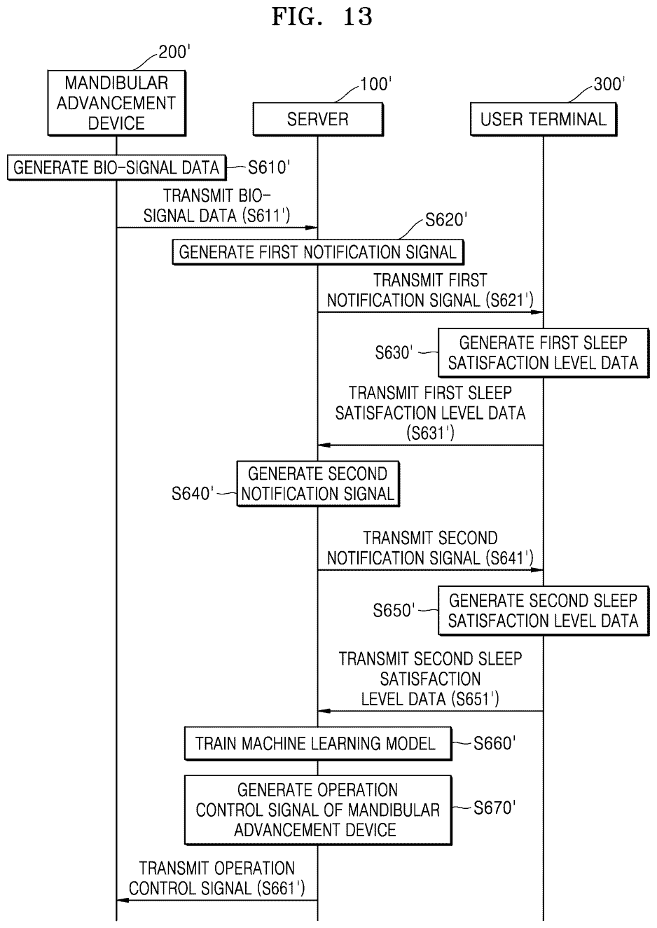

FIG. 13 is a flowchart for explaining a control method of a mandibular advancement system.

Referring to FIG. 13, in operation S610′, the sleep disorder treatment device 200′ generates bio-signal data during a user's sleep by using a sensing unit. The sleep disorder treatment device 200′ may transmit the bio-signal data detected in real time to the sleep disorder control device 100′, or transmit bio-signal data detected when a sleep disorder event has occurred, or transmit bio-signal data sensed at each preset cycle in operation S611′.

In operation S620′, the sleep disorder control device 100′ generates a first notification signal after the user wakes up after completing sleep. The sleep disorder control device 100′ may generate a first notification signal at a preset time point, or may detect a user's waking up by using bio-signal data and generate a first notification signal. The sleep disorder control device 100′ transmits the generated first notification signal to the user terminal 300′ in operation S621′.

In operation S630′, the user terminal 300′ provides questionnaire information including a sleep satisfaction level-related questionnaire through an interface, and generates first sleep satisfaction level data at a first time point by using response information according to the user's selection. The first sleep satisfaction level data may further include personal information of the user. The user terminal 300′ transmits the first sleep satisfaction level data to the sleep disorder control device 100′ in operation S631′.

However, the present disclosure is not limited thereto, and as another embodiment, when the machine learning model trained by the sleep disorder control device 100′ is provided to the sleep disorder treatment device 200′, the sleep disorder control device 100′ may transmit the first sleep satisfaction level data to the sleep disorder treatment device 200′. In other words, the sleep disorder control device 100′ may train the machine learning model by using previous first sleep satisfaction level data as learning data, and the sleep disorder treatment device 200′ may control the operation of the sleep disorder treatment device 200′ by using the trained machine learning model and also by additionally using the new first sleep satisfaction level data.

In operation S640′, the sleep disorder control device 100′ generates a second notification signal at a time point different from that at which the first notification signal is generated. The second notification signal may be generated before the user spends the day and goes to sleep. The sleep disorder control device 100′ may generate the second notification signal before the average time when the user sleeps, or may generate the second notification signal at a preset time point. The sleep disorder control device 100′ transmits the second notification signal to the user terminal 300′ in operation S631′.

In operation S650′, the user terminal 300′ provides questionnaire information including a sleep satisfaction level-related questionnaire through an interface, and generates second sleep satisfaction level data at a second time point different from the first time point, by using response information according to the user's selection. The second time point may be after a preset period of time from the first time point and before a next sleep of the user, and the user may enter state information about daytime sleepiness, concentration, work efficiency, etc. through the user terminal 300′. The user terminal 300′ transmits the second sleep satisfaction level data to the sleep disorder control device 100′ in operation S651′.

In operation S660′, the sleep disorder control device 100′ may train the machine learning model based on the sleep satisfaction level data, the bio-signal data, and the usage record data. The machine learning model may be an algorithm for learning control criteria for controlling the operation of the sleep disorder treatment device 200′ based on the sleep satisfaction level data, the bio-signal data, and the usage record data.

In operation S670′, the sleep disorder control device 100′ generates an operation control signal for controlling the operation of the sleep disorder treatment device 200′ by applying the sleep satisfaction level data, the bio-signal data, and the usage record data to the trained machine learning model. The server 100′ may transmit the generated operation control signal to the sleep disorder treatment device 200′ in operation S661′ to control the sleep disorder treatment device 200′.

As described above, according to the sleep disorder control device and method according to the embodiments of the present disclosure, a sleep disorder is detected using biometric information, and when treating a detected sleep disorder by advancing the mandible, sleep quality may be improved by minimizing arousal due to the movement of the mandible by also considering the sleep satisfaction level of the user. According to the sleep disorder control device and method of the embodiments of the present disclosure, the learning efficiency may be improved by using, as learning data, not only sleep satisfaction level data obtained immediately after sleep but also sleep satisfaction level data obtained before going to sleep after spending a day in daily life.

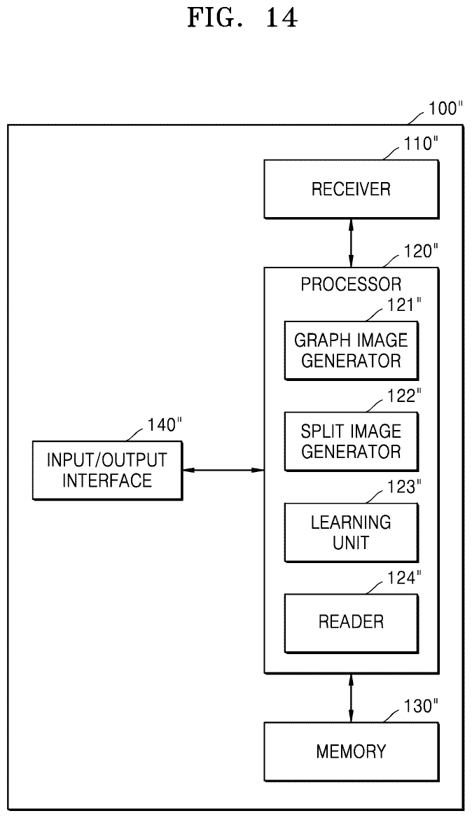

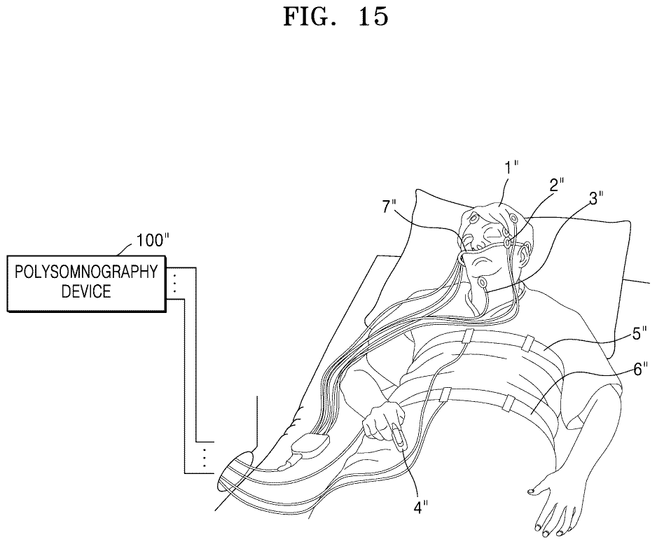

FIG. 14 is a block diagram schematically illustrating a polysomnography device 100″ according to an embodiment of the present disclosure, and FIG. 15 is a conceptual diagram for explaining a process of obtaining polysomnography data from a plurality of examination units.

Referring to FIGS. 14 and 15, the polysomnography device 100″ according to an embodiment of the present disclosure may obtain polysomnography data from external examination units 1″ to 7″, and generate learning data by using the polysomnography data, and then may effectively train a sleep state reading model based on the generated learning data.

A network environment according to the present disclosure may include a plurality of user terminals, a server, and a network. The polysomnography device 100″ may be a server or a user terminal.

The plurality of user terminals may be stationary terminals implemented by a computer device or mobile terminals. When the polysomnography device 100″ is a server, the plurality of user terminals may be terminals of an administrator who controls the server. For example, the plurality of user terminals may include smart phones, smart watches, mobile phones, navigation devices, computers, laptop computers, digital broadcasting terminals, Personal Digital Assistants (PDA), Portable Multimedia Players (PMD), tablet PCs, etc. For example, the user terminals may communicate with other user terminals and/or a server through a network by using a wireless or wired communication method.

The communication method is not limited, and not only a communication method using a communication network that the network may include (e.g., a mobile communication network, a wired Internet, a wireless Internet, a broadcasting network), but also short-range wireless communication between devices may be included as the communication method. For example, the network may include one or more of a personal area network (PAN), a local area network (LAN), a controller area network (CAN), a metropolitan area network (MAN), a wide area network (WAN), a metropolitan area network (MAN), a wide area network (WAN), a broadband network (BBN), and the Internet. Further, the network may include any one or more of network topologies including a bus network, a star network, a ring network, a mesh network, a star-bus network, a tree or a hierarchical network, and the like, but is not limited thereto.

The server may be implemented using a computer device that communicates with a plurality of user terminals through a network to provide commands, codes, files, contents, services, and the like, or a plurality of the computer devices.

For example, the server may provide a file for installing an application to a user terminal accessed through a network. In this case, the user terminal may install the application by using a file provided from the server. In addition, according to the control by an operating system (OS) and at least one program (e.g., a browser or an installed application) included in the user terminal, the user terminal may access the server to receive services or contents provided by the server. As another example, the server may establish a communication session for data transmission or reception, and route data transmission or reception between the plurality of user terminals through the established communication session.

Meanwhile, the polysomnography device 100″ may include a receiver 110″, a processor 120″, a memory 130″, and an input/output interface 140″.

The receiver 110″ may receive polysomnography data from the external examination units 1″ to 7″. As an embodiment, the receiver 110″ of the polysomnography device 100″ may be connected to the external examination units 1″ to 7″ by wires as illustrated in FIG. 15 and obtain polysomnography data measured in time series. In another embodiment, the receiver 110″ may function as a communication module using wireless communication and receive polysomnography data.

The polysomnography data may be a plurality of pieces of biometric data of a user, measured using a plurality of examination units. The plurality of pieces of biometric data may include biometric data obtained using at least one of sensing units among an Electroencephalogram (EEG) sensor, an Electrooculography (EOG) sensor, an Electromyogramme (EMG) sensor, an Electrokardiogramme (EKG) sensor, a Photoplethysmography (PPG) sensor, a chest belt, an abdomen belt, oxygen saturation, end-tidal CO2 (EtCO2), a respiration detection thermistor, a flow sensor, a pressure sensor (manometer), a microphone, and a positive pressure gauge of a continuous positive pressure device.

In detail, the plurality of pieces of biometric data may include at least one of biometric data related to brain waves from the EEG sensor 1″, biometric data related to eye movement from the EOG sensor 2″, biometric data related to muscle movement from the EMG sensor 3″, biometric data related to a heart rate from an EKG sensor (not shown), biometric data related to oxygen saturation and a heart rate from the PPG sensor 4″, biometric data related to movement of the abdomen and the chest from the chest motion detection belt 5″ and the abdominal motion detection belt 6″, biometric data related to respiration, from EtCO2, the respiration detection thermistor, and the flow sensor 7″, and biometric data related to snoring, from a microphone (not shown). In addition, the plurality of pieces of biometric data may include positive pressure level data obtained using the positive pressure gauge of a continuous positive pressure device.

The processor 120″ may be configured to process a command of a computer program by performing basic arithmetic, logic, and input/output operations. The command may be provided to the processor 120″ by the memory 130″ or the receiver 110″. For example, the processor 120″ may be configured to execute received commands according to program code stored in a recording device, such as the memory 130″. Here, the ‘processor’ may refer to, for example, a data processing device embedded in hardware and having a physically structured circuit to perform a function expressed as code or a command included in a program.

Examples of the data processing device embedded in hardware as described above may include a microprocessor, a central processing unit (CPU), a processor core, a multiprocessor, an application-specific integrated circuit (ASIC), and a field programmable gate array (FPGA), but the present disclosure is not limited thereto. The processor 120″0 may include a graph image generator 121″, a learning unit 123″, and a reader 124″, and may further include a split image generator 122″.

The memory 130″ is a computer-readable recording medium and may include a random access memory (RAM), a read only memory (ROM), and a permanent mass storage device such as a disk drive. In addition, the memory 130″ may store an operating system and at least one program code (e.g., code for a browser installed and driven in a user terminal or the application described above). These software components may be loaded from a computer-readable recording medium that is readable by an additional computer, separate from the memory 130″ by using a drive mechanism. The computer-readable recording medium readable by an additional computer may include a computer-readable recording medium such as a floppy drive, a disk, a tape, a DVD/CD-ROM drive, and a memory card. In another embodiment, the software components may be loaded into the memory 130″ through the receiver 110″ instead of a computer-readable recording medium. For example, at least one program may be loaded to the memory 130″ based on a program (e.g., the application described above) installed by files provided by, through a network, a file distribution system (e.g., the server described above) for distributing installation files of developers or applications.

The input/output interface 140″ may be used for interfacing with an input/output device. For example, an input device may include a device such as a keyboard or mouse, and an output device may include a device such as a display for displaying a communication session of an application. As another example, the input/output interface 140″ may be used for interfacing with a device in which functions for inputting and outputting are integrated into one, such as a touch screen.

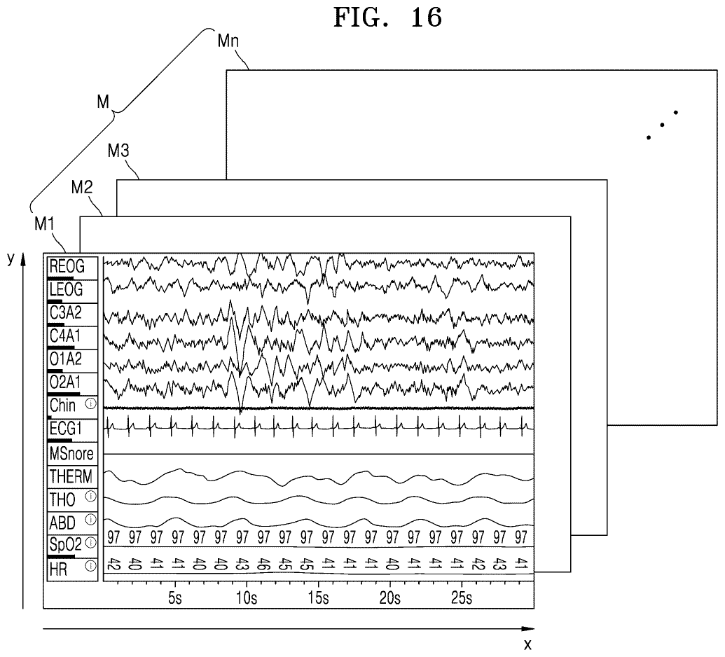

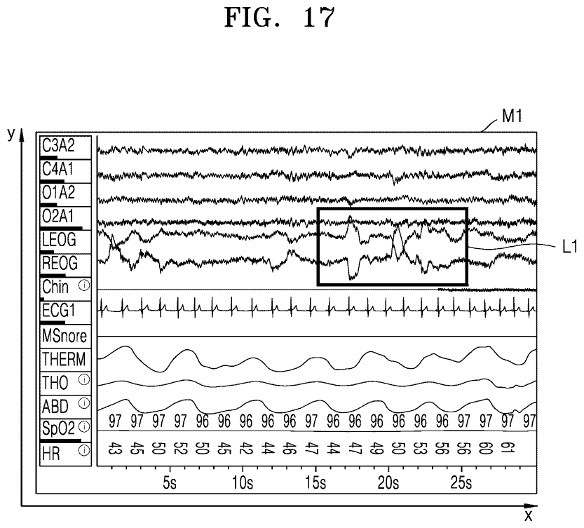

Hereinafter, the polysomnography device 100″ will be described in detail with further reference to FIGS. 16 and 17.

FIG. 16 is a diagram illustrating a graph image that is learning data of the polysomnography device 100″ according to an embodiment of the present disclosure, and FIG. 17 is a diagram illustrating a labeled graph image.

Referring back to FIG. 14 and FIGS. 16 and 17, the polysomnography device 100″ includes the graph image generator 121″, the learning unit 123″, and the reader 124″, and may further include the split image generator 122″. Here, the polysomnography device 100″ may include a single processor including the components described above, but may include the above-described components by using two or more processors. For example, the learning unit 123″ of the polysomnography device 100″ may be included in a processor of a server, and the reader 124″ may be included in a processor of a user terminal. That is, the polysomnography device 100″ may transmit biometric data of a user to the server in which the learning unit 123″ is arranged, and train a sleep state reading model, and may transmit the trained sleep state reading model to the reader 124″ of the user terminal to perform a function of reading a newly measured sleep state of the user.

The graph image generator 121″ may obtain polysomnography raw data measured in time series, and convert the raw data into a graph with respect to time, to generate a graph image M. As an embodiment, the graph image generator 121″ may convert each of a plurality of pieces of biometric data into individual graphs with respect to time, and sequentially arrange the converted, plurality of individual graphs on a time axis (e.g., x-axis) and generate the graph image M. In other words, the plurality of detection units 1″ to 7″ may obtain biometric data in time series, and a data value of the biometric data may change over time. The graph image generator 121″ may convert each piece of biometric data into a graph represented by a change in the data value over time, and output each graph as a single image. Here, the graph image generator 121″ may generate a graph image by matching times of a plurality of pieces of biometric data. The plurality of pieces of biometric data converted into individual graphs may be sequentially arranged on a time axis. Types of each piece of biometric data may be displayed on a y-axis intersecting with the time axis (x-axis) of the graph image M, but the present disclosure is not limited thereto. In addition, the graph image generator 121″ may obtain a plurality of pieces of biometric data as raw data and convert the same into a certain format, and then generate a graph image. The graph image generator 121″ may generate a graph image in a certain format regardless of the type of detection unit, the combination of detection units, and the configuration by component manufacturing companies. Thereafter, the learning unit 123″ may train a standardized sleep state reading model by using the graph image of the certain format as learning data.

Also, the graph image M may include labeled data. As illustrated, as a labeling method, a labeling method using bounding boxes as illustrated, a labeling method using scribbles, a labeling method using points, an image-level labeling method, etc. may be used. A label L1 may be information indicating a sleep state that is read and displayed in advance by a professional examination personnel. The sleep state may include at least one of sleep stages such as W (wake stage), N1 (sleep stage 1), N2 (sleep stage 2), N3 (sleep stage 3), R (REM sleep stage), a sleep apnea state, a snoring state, an oxygen saturation-reduced state.

The split image generator 122″ may generate a plurality of images M1, M2, . . . , Mn (see FIG. 16) by splitting the graph image M in units of a preset time. In the present disclosure, the graph image M may be used as learning data, but the graph image M may also be split into the images M1, M2, . . . Mn described above and used as learning data. The images M1, M2, . . . Mn may be a set of pieces of biometric data commonly required to interpret a certain stage or certain state of sleep. In this case, the preset time unit may be a unit displayed as one screen on a display device during polysomnography; for example, a graph image may be split in units of 30 seconds. In this case, since the images M1, M2, . . . Mn are biometric data measured time-serially overnight, they may have serial characteristics.

As another embodiment, the images obtained by splitting the graph image M may be generated by extracting a graph area of each detection unit from the graph image M. That is, the polysomnography device 100″ may use, as learning data, one graph image M in which a plurality of pieces of biometric data are displayed, but may also generate a graph image for each piece of biometric data and use the same as learning data.

As another embodiment, the graph image M may be a captured image of a screen displayed on an external display device. That is, the polysomnography device 100″ may not separately obtain biometric data, but may be linked to a display device and capture a graph displayed on the screen for each preset time unit and generate a graph image. In this case, the polysomnography device 100″ may further include a pre-processing unit (not shown). The pre-processing unit (not shown) may convert formats for a scale (size, resolution), contrast, brightness, color balance, and hue/saturation of a graph image in order to maintain the consistency of captured images.

The learning unit 123″ may train a sleep state reading model based on the graph image M described above. When the graph image M includes a plurality of images obtained by splitting the graph image M, the learning unit 123″ may train the sleep state reading model based on the plurality of images.

The sleep state reading model may be a learning model for reading at least one of sleep apnea syndrome, periodic limb movement disorder, narcolepsy, sleep stages, and total sleep time. The learning unit 123″ trains a sleep state reading model based on deep learning or artificial intelligence, and deep learning is defined by a machine learning algorithm that tries high-level abstractions (summarizing key contents or functions from large amounts of data or complex data) through a combination of non-linear transformation methods. The learning unit 123″ may use, among deep learning models, for example, one of a deep neural network (DNN), a convolutional neural network (CNN), a recurrent neural network (RNN), and a deep belief neural network (DBN).

As an embodiment, the learning unit 123″ may train a sleep state reading model by using a convolutional neural network (CNN). Here, a convolutional neural network (CNN) is a type of multilayer perceptrons designed to use minimal preprocessing. A convolutional neural network (CNN) includes a convolutional layer that performs convolution on input data, and may further include a subsampling layer that performs subsampling on an image, and thus extract a feature map from the data. Here, the subsampling layer is a layer that increases the contrast between neighboring data and reduces the amount of data to be processed, and max pooling, average pooling, etc. may be used.

Each of the convolutional layers may include an activation function. The activation function may be applied to each layer to perform a function of making each input have a complex non-linear relationship. As the activation function, a sigmoid function, a tanh function, a Rectified Linear Unit (ReLU), a Leacky ReLU, etc., which are capable of converting an input into a normalized output may be used.

The reader 124″ may read a sleep state of a user who is a subject of examination, based on the graph image of the subject and the trained sleep state reading model. The reader 124″ may directly receive a graph image rather than measured source data from the examination units, and apply the graph image to the sleep state learning model to read the user's sleep state. In addition, the reader 124″ may output and provide the read sleep state of the user as a result.

The polysomnography device 100″ may receive feedback on a reading result derived using the sleep state reading model, generate feedback data therefor, and provide the feedback data to the learning unit 123″. The learning unit 123″ may re-train the sleep state reading model by using the feedback data, thereby deriving a more accurate reading result.

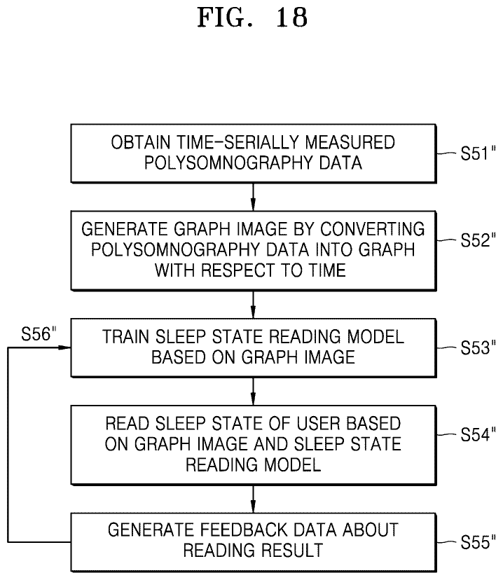

FIG. 18 is a diagram sequentially illustrating an examination method of a polysomnography device according to an embodiment of the present disclosure.

Referring to FIG. 18, the polysomnography device 100″ may obtain, by the receiver 110″, time-serially measured polysomnography data in operation S51″.

In operation S52″, the polysomnography device 100″ may generate a graph image by converting the polysomnography data into a graph with respect to time by using the graph image generator 121″. The graph image may be split in units of a preset time unit and converted into images obtained by splitting the graph image.

In operation S53″, the polysomnography device 100″ may train the sleep state reading model based on the graph image by using the learning unit 123″. When the graph image includes the plurality of images obtained by splitting the graph image, the learning unit 123″ may train the sleep state reading model based on the plurality of images.

In operation S54″, the polysomnography device 100″ may read, by using the reader 124″, the sleep state of the user based on the graph image and the sleep state reading model. The graph image here may be an image processed using a plurality of pieces of biometric data obtained from a plurality of examination units. Alternatively, the graph image may be an image obtained by capturing a graph displayed on the screen of the display device for monitoring polysomnography.

In operation S55″, the polysomnography device 100″ may receive feedback on the reading result of the reader 124″, and generate feedback data thereof. Feedback on the reading result may be performed by a professional polysomnography personnel, and the learning unit 123″ may derive an accurate reading result by re-training the sleep state reading model by using the feedback data.

As described above, according to the polysomnography device and method according to the embodiments of the present disclosure, instead of raw data obtained from a plurality of examination units, a graph image generated using the raw data is used as learning data, thus allowing to derive accurate reading results while increasing the learning efficiency based on artificial intelligence or deep learning. According to the polysomnography device and method according to the embodiments of the present disclosure, automation of the examination may be realized through the trained sleep state reading model, thereby shortening the examination time as well as reducing examination deviation according to a reader. In addition, the polysomnography device and method according to the embodiments of the present disclosure may also be used as a convenient and continuous sleep monitoring apparatus because algorithms are used in various daily IT products such as smart watches.

The embodiments according to the present disclosure described above may be implemented in the form of a computer program that can be executed through various components on a computer, and such a computer program may be recorded in a computer-readable medium. The medium may store a computer-executable program. Examples of the medium include magnetic media such as a hard disk, a floppy disk, and a magnetic tape, optical recording media such as CD-ROM and DVD, magneto-optical media such as a floptical disk, and those configured to store program instructions, including ROM, RAM, flash memory, and the like.

The computer program may be specifically designed and configured for the embodiments of the present disclosure or may be well-known and available to one of ordinary skill in the art Examples of the program instructions include not only machine codes generated by using a compiler but also high-level language codes that can be executed on a computer by using an interpreter or the like.

While the present disclosure is described with reference to the embodiments illustrated in the drawings, this is merely exemplary, and those of ordinary skill in the art will understand that various modifications and equivalent other embodiments may be made therefrom. Therefore, the scope of the present disclosure shall be defined by the appended claims.

According to an embodiment of the present disclosure, a respiratory status examination apparatus and method and a sleep disorder control device and method are provided. In addition, embodiments of the present disclosure may be applied to industrially used examination and treatment of sleep disorders.

What is claimed is:

1. A sleep disorder control method comprising:
obtaining sleep satisfaction level data and bio-signal data of a user wearing a sleep disorder treatment device, and usage record data of the sleep disorder treatment device;
training a machine learning model based on the sleep satisfaction level data, the bio-signal data, and the usage record data; and
controlling an operation of the sleep disorder treatment device while the user is wearing the sleep disorder treatment device by using the sleep satisfaction level data, the bio-signal data, the usage record data, and the machine learning model.

2. The sleep disorder control method of claim 1, wherein obtaining the sleep satisfaction level data, the bio-signal data, and the usage record data comprises:
obtaining the bio-signal data of the user and the usage record data of the sleep disorder treatment device during a sleep of the user wearing the sleep disorder treatment device; and
obtaining the sleep satisfaction level data after the user wearing the sleep disorder treatment device completes the sleep.

3. The sleep disorder control method of claim 2, wherein the obtaining the sleep satisfaction level data comprises:
obtaining first sleep satisfaction level data at a first time point when the user completes the sleep; and
obtaining second sleep satisfaction level data at a second time point different from the first time point.

4. The sleep disorder control method of claim 3, wherein the obtaining the second sleep satisfaction level data comprises:
obtaining the second sleep satisfaction level data after a preset period of time from the first time point and before a next sleep of the user.

5. The sleep disorder control method of claim 3, wherein the obtaining the sleep satisfaction level data comprises:
generating a first notification signal to the user before the first time point; and
generating a second notification signal to the user before the second time point.

6. The sleep disorder control method of claim 1, wherein the controlling the operation of the sleep disorder treatment device comprises:
controlling a degree of advancement or a number of advances of the sleep disorder treatment device while the user is wearing the sleep disorder treatment device.

7. A sleep disorder control device comprising:
a data obtaining unit configured to obtain sleep satisfaction level data and bio-signal data of a user wearing a sleep disorder treatment device, and usage record data of the sleep disorder treatment device;
a learning unit configured to train a machine learning model based on the sleep satisfaction level data, the bio-signal data, and the usage record data; and
an operation controller configured to control an operation of the sleep disorder treatment device while the user is wearing the sleep disorder treatment device by using the sleep satisfaction level data, the bio-signal data, the usage record data, and the machine learning model.

8. The sleep disorder control device of claim 7, wherein the data obtaining unit comprises:
a bio-signal obtaining unit configured to obtain the bio-signal data by using one or more sensors during a sleep of the user wearing the sleep disorder treatment device;
a usage record obtaining unit configured to obtain the usage record data of the sleep disorder treatment device during the sleep of the user wearing the sleep disorder treatment device; and
a sleep satisfaction level obtaining unit configured to obtain the sleep satisfaction level data after the user wearing the sleep disorder treatment device completes the sleep.

9. The sleep disorder control device of claim 8, wherein the sleep satisfaction level obtaining unit is configured to:
obtain first sleep satisfaction level data at a first time point when the user completes the sleep; and
obtain second sleep satisfaction level data at a second time point different from the first time point.

10. The sleep disorder control device of claim 9, wherein the second sleep satisfaction level data is obtained at the second time point after a preset period of time from the first time point and before a next sleep of the user.

11. The sleep disorder control device of claim 9, further comprising:
a notification signal generator configured to generate a first notification signal to the user before the first time point and to generate a second notification signal to the user before the second time point.

12. The sleep disorder control device of claim 7, wherein the operation controller is configured to control, by using the sleep satisfaction level data, the bio-signal data, the usage record data, and the machine learning model, a degree of advancement or a number of advances of the sleep disorder treatment device while the user is wearing the sleep disorder treatment device.
